(19) United States
(12) Patent Application Publication
(10) Pub. No.: US 2024/0293285 A1
(43) Pub. Date: Sep. 5, 2024

(54) OPENING DEVICE FOR ASEPTIC CARTON PACKAGING USED FOR ENTERAL NUTRITION THROUGH CLOSED SYSTEM

(71) Applicant: Ronaldo SANTOS LEITE, Paulínia/SP (BR)

(72) Inventor: Ronaldo SANTOS LEITE, Paulínia/SP (BR)

(21) Appl. No.: 18/662,915

(22) Filed: May 13, 2024

Related U.S. Application Data
(63) Continuation of application No. PCT/BR2022/050434, filed on Nov. 11, 2022.

(30) Foreign Application Priority Data
Nov. 13, 2021 (BR) 1020210229144

(51) Int. Cl. A61J 1/14

(52) U.S. Cl. CPC A61J 1/1412

(57) ABSTRACT

The present invention falls within the technical field of Food Packaging and has been specifically created to cater to the needs of Enteral Nutrition Foods Administered by a Closed System, defining itself as an opening device that is part of an aseptic carton packaging for exclusive use through connection to the enteral feeding set. Its design redefines traditional aseptic carton packaging (used in open enteral systems) for a closed enteral system packaging, as its features allow for the infusion of the product through the enteral feeding tube and preserve the packaging's barriers and airtightness without exposing the product to the environment, eliminating handling and the consequent need for a specific preparation area.

This application is a Continuation of International Patent Application No. PCT/BR2022/050434, filed on Nov. 11, 2021, which claims benefit of priority to patent application Ser. No. 10/202,10229144, filed on Nov. 13, 2021 in Brazil, and which applications are incorporated herein by reference. To the extent appropriate, a claim of priority is made to each of the above disclosed applications.

### FIELD OF THE INVENTION

The present invention relates to the field of opening devices found in aseptic carton packaging used for food. It is defined as an opening device consisting of a top part (cap) and a bottom part (base) equipped with an insert for connecting to enteral feeding sets. Since it is inseparably integrated into the packaging during the manufacturing stage, this opening device determines the function of the packaging and the way to use its stored contents. By replacing the opening devices traditionally used in aseptic carton packaging, this invention enables the administration of these products in closed enteral systems.

### BACKGROUND OF THE INVENTION

Nutritional therapy (NT), an essential strategy in patient care for both humans and animals, can be implemented in three modalities: oral, enteral, and parenteral. Parenteral nutritional therapy (PNT), which is not relevant to the state of the art for this invention, involves the intravenous administration of nutrients. It is recommended for patients whose digestive tract is entirely compromised or as a supplement for those unable to meet their nutritional needs exclusively through the digestive system. On the other hand, oral feeding, also unrelated to the state of the art of the present invention, is the preferred option for patients with a functionally intact digestive system, allowing normal ingestion of food orally.

Enteral nutritional therapy (ENT), for which this invention was developed, is a method of providing nutrients through the gastrointestinal tract (GIT). It is typically used when the patient has a functional GIT but cannot consume food orally to meet their nutritional needs.

ENT is indicated for cases where oral intake of food is insufficient or impossible, but the gastrointestinal tract is fully or partially functional. This may occur for patients with neurological damage, such as a stroke, patients with swallowing difficulties, or those in a reduced state of consciousness due to surgeries or other health complications.

Enteral nutrition offers several advantages over parenteral nutrition, such as using the gastrointestinal tract, which helps maintain intestinal barrier function and supports the gut microbiome, among other benefits.

### About Enteral Nutrition Administration Systems

Enteral nutrition is a process that involves the use of an administration system, consisting of an enteral feeding tube and a device known as an enteral feeding set, which connects the tube to the nutritional product.

In enteral administration, the feeding tube and feeding set play fundamental roles. The enteral feeding set, which is the primary device relevant to the present invention, comprises a flexible tube with a sharp element at the proximal end designed to penetrate a specific region of the container holding the enteral food, along with other elements that work synergistically to form a secure connection with the container.

The part of the enteral system formed by the feeding set connected to the enteral feeding tube provides an efficient and secure channel for administering the food, allowing the enteral formula to be delivered directly to the clinically appropriate portion of the patient's digestive system, whether in the stomach or intestine. This procedure ensures that the patient's nutritional needs are met, even when health conditions prevent conventional oral feeding.

Regarding enteral feeding administration techniques, two systems are used, each with unique characteristics directly related to maintaining product sterility and offering different levels of safety during handling, preparation, and administration to the patient: the open system and the closed system. It is important to note that the specifics of the administration technique through the closed system are closely related to the distinguishing feature of the present invention.

### About Enteral Nutrition Administered Via an Open System:

The most common way to administer enteral nutrition using an open system involves the use of disposable plastic bottles specifically developed for this purpose. These bottles are equipped with caps adapted to connect to enteral feeding sets designed for enteral nutrition administration, allowing the product to be delivered directly to the patient through an feeding tube.

In the open system, the industrially produced enteral nutrition formula is transferred from its original packaging, an aseptic carton packaging, into the enteral feeding bottle or bag. This transfer can be done manually or with the aid of devices that facilitate the process. It's important to note that aseptic carton packaging used in open-system products typically features automated opening devices (AODs).

However, when the formula is transferred from the aseptic carton packaging to the enteral feeding container, it becomes exposed to the environment and loses its sterility. For this reason, the open system has a shorter administration timeframe after preparation—typically 4 to 8 hours from opening to the end of infusion—and requires careful handling to ensure patient safety. This is a significant disadvantage compared to the closed system, which has an administration timeframe of 24 to 48 hours. It's mandatory that the handling of the enteral nutrition formula in the open system be performed in a hygienically controlled and validated environment.

The integrated opening system commonly used in aseptic carton packaging generally consists of an automated opening device—composed of a base with a spout and a cap—affixed to a pre-cut and weakened section of the packaging. Such automated opening devices are incorporated into aseptic carton packaging by an automated machine. This machine applies a layer of hot-melt adhesive to the underside of the base of the opening device, then positions it over the weakened section of the packaging, firmly pressing it down. The hot-melt adhesive cools rapidly, integrating the opening device to the packaging and completing the process.

It is important to note that such applicator machines are highly efficient and accurate, capable of processing a large volume of packaging per minute in a controlled environment. Furthermore, these machines are pre-set to apply a device of a particular size and shape. Therefore, it is crucial that the opening device is made from a material robust enough to withstand the machine's forces during processing and is precisely dimensioned to match the applicator machine's pre-established parameters. It's worth noting that this invention was developed to work seamlessly with these machines without requiring any adjustments to the pre-set parameters for the standard opening device.

The importance of barrier layers in food packaging is widely recognized, essential for protecting them against the detrimental effects of oxygen and light. Aseptic carton packaging is a notable example of this type of packaging. Long before its application in enteral formulas, this packaging was already used to aseptically contain liquid products.

Such multilayer packaging typically features a thicker layer of fibrous material that is coated on both sides by layers of a sealing material, usually made of low-density polyethylene (LDPE). Thus, during the packaging formation, both surfaces (external and internal) are covered by layers of this sealing material. The thicker fibrous material, usually made of cardboard, provides structural stability to the packaging. For this reason, these packages are commonly known as aseptic carton packaging.

Over the years, these packages have undergone evolutions, especially in their opening systems. Such systems are widely recognized and used across a broad range of products, from items like milk and juices to enteral nutrition (open system). Some of these mechanisms include: a) packaging without an integrated opening system (WIOS); b) “Single Serving” opening system (SSOS); c) “Pull Tab” opening system (PTOS); d) “ReCap” type cap (RCTL); e) cap with manual seal opening (LMSO); f) automated opening device (AOD). The latter, the automated opening device (AOD), will be particularly detailed. This is due to the fact that the process of applying the AOD is identical to the process of applying the device conceived by the present invention, developed to be applied as a replacement for the AOD when the manufactured product is intended for a closed system, justifying its importance to this document.

The automated opening device (AOD) is commonly integrated into the top of aseptic multilayer packaging, consisting of a base with a threaded spout and a corresponding cap. This configuration allows the packaging to be resealed and stored for future use after it has been opened and partially used.

The laminated material intended for this type of opening has the top layers of plastic and cardboard strategically removed in the region just below where the AOD will be affixed. The resulting region is a weakened area, a circular cut concentric to the spout of the AOD's base, which acts as a protective seal for the contents. It is precisely this weakened layer that will be cut during the first opening of the cap.

Within the AOD, positioned inside the base's spout in a cooperative manner relative to the cap, there is a cutting element, typically cylindrical in shape with a serrated lower end, which is activated upon the first rotation of the cap, cutting through the weakened region of the packaging and opening it.

It is widely known that enteral nutrition products intended for open systems use aseptic carton packaging. Thus, the techniques related to the open system present a mode of administration that requires opening the packages and transferring the product to bottles developed for this purpose.

The patent literature indicates that alternative solutions have been proposed to allow the administration of enteral products directly from their original aseptic carton packaging, using specially adapted enteral feeding sets to connect to the spouts of these packages. These solutions can be seen in document CN1711116A and patent application BR2020210228593 filed with INPI-BR. However, the practical implementation of this technique is limited and not widely adopted.

Other solutions proposed in the patent literature that enable the administration of enteral products directly from their original aseptic packaging include adapters or external connectors equipped with an access point for enteral feeding sets, which can be screwed onto the existing spout of aseptic carton packaging or affixed to another part of this packaging. Examples of such devices are illustrated in patent documents WO2019193213A1, EP1352843A1, and CN1711116A.

Patent document WO2019193213A1, unlike an opening device, describes an adapter intended to attach to the spout present in an aseptic carton package. It provides multiple access points to which various devices can be connected for specific functions: mixer, applicator, and adapter. The latter is an adapter comprising a device equipped with an access point to connect enteral feeding sets and is designed to be screwed onto the spout of the packaging after its primary cap has been removed.

Patent document EP1352843A1, unlike an opening device, describes an external adapter designed to be adhered to an aseptic carton package that has a straw access point, similar to a package with a “Single Serving” (SASS) opening system. The method specifies that the opening in the adapter should be positioned concentrically with the opening in the aseptic multilayer package. The document describes an external device consisting of two separate modules that together provide a means of administering enteral products directly from their original packaging.

Patent document CN1711116A, unlike an opening device, describes a connector intended for use with an enteral feeding set and a laminated paper packaging system. According to CN1711116A, this packaging system comprises an aseptic carton package already equipped with an opening system that includes a spout. Among the methods presented in the document, the most relevant technique involves designs intended to be screwed onto the spout of an aseptic carton package (like Tetra Brik®). It's crucial to highlight that the description clearly demonstrates that this is an external device functioning as an additional accessory, not an integral or permanent part of the packaging. This aspect is evident since the device needs to be screwed onto the spout of the package to open it and access the product. The technique specified in CN1711116A requires the connector to be screwed onto the spout in a sealing direction, breaking the seal and accessing the contents. This action of screwing the connector onto the spout and thereby breaking the protective seal clearly demonstrates the external nature of the device. In contrast, automated opening devices on aseptic carton packages open the packaging when the cap is unscrewed from the spout, breaking a tamper-evident seal and cutting through the protective seal of the contents. This difference in opening techniques, besides highlighting the external nature of the connector, contradicts the principle of a closed enteral system because when the protective barrier provided by the seal is breached, the package contents are exposed to the environment. Therefore, the device described in CN1711116A is not an opening device already present in an aseptic carton package, and the associated technique does not meet the requirements necessary for administering its contents in a closed enteral system.

A comprehensive analysis of the devices suggested in the current state of the art reveals that: a) the opening devices currently used in multilayer aseptic packaging are not suitable for enteral administration, requiring additional external devices; b) the additional external devices used to administer enteral products directly from their aseptic carton packaging are external elements designed to fit the spouts present in the opening devices of such packaging. Furthermore, the techniques described for using these external devices share a vulnerability incompatible with closed enteral systems: the need to open the packaging before connecting the enteral feeding set. This process inevitably exposes the contents to the environment, compromising the product's sterility.

In the practice of enteral nutritional therapy, the use of aseptic carton packaging has been limited to open enteral systems, revealing a gap in current technology. This observation indicates the opportunity for a new concept of an opening device for aseptic carton packaging that retains similarities with existing opening devices but is equipped with a means of access for standard enteral feeding sets.

### About Enteral Nutrition Administered Via a Closed System:

The closed system of enteral nutrition is an approach that eliminates the need for handling, transferring, or adding components to the formulas. This is because the enteral nutrition formula is already prepared, sealed, and sterilized in its packaging, which is designed to maintain sterility throughout the entire process, from manufacturing to administration to the patient.

In the closed enteral nutrition system, the product's packaging is meticulously designed to ensure that its contents do not come into contact with the external environment at any point until the product is fully administered to the patient. This system, which keeps the product completely protected until it is fully infused, is made possible by the caps of these packages, which are specially designed with devices for direct connection to the enteral nutrition set. Some examples of these access devices are disclosed in patents WO2010030528 and US2018296438A1. In a closed system, access to the product occurs only when the enteral set penetrates the packaging, breaking the protective seal while simultaneously sealing the packaging with the set's own devices that fit snugly into the cap devices. This connection must occur in a way that the interior of the packaging is not exposed to the external environment, not even for a moment.

Current packaging for closed-system products can be categorized as non-collapsible (more rigid) or collapsible (more flexible).

Non-collapsible packaging is equipped with caps that incorporate both an access device for tubing and microbiological filters. These filters enable air from the environment to neutralize the internal pressure of the packaging, thus allowing the infusion of the contents by gravity flow. Examples of these air inlet devices can be found in caps designed for rigid packaging intended for closed-system products, as evidenced by patents WO2010030528A2, U.S. Pat. No. 20,182,96438A1, and commercial products with designs similar to the technical drawings U.S. D427306S and U.S.D330332S.

On the other hand, collapsible packaging eliminates the need for air intake provided by such filters. Due to their highly flexible nature, the structure of these packages does not exhibit resistance to the negative internal pressure generated during the infusion process, ensuring a uniform flow of the contents.

Regarding the types of access available in containers for administering enteral nutrition and their corresponding feeding sets:

In containers that store enteral nutrition products prior to administration, both disposable bottles used for portioning open-system products and the packaging for closed-system products commonly feature two types of set connections: a connection for spike-type sets and a connection for ENFit-type sets.

The spike enteral feeding set is characterized by the design of the device at its proximal end, which gradually tapers to resemble a thorn or spear. This device has a sharp tip that, when inserted into the designated access area, punctures the packaging of the enteral nutrition product.

The ENFit enteral feeding set represents a connection type with a more specific design, established as an international standard for enteral connections. This standard aims to enhance patient safety, prevent administration errors, and ensure compatibility among various medical devices. The ENFit connection also has a pointed tip to puncture the designated area of the packaging but is distinguished by its cross shape, which provides a more precise fit with the container holding the product. Additionally, a female thread surrounds this connection, preventing accidental disconnections and ensuring secure administration of enteral nutrition.

Such mechanisms, together with the enteral feeding tube and the container holding the enteral formula, form an enteral system. This system allows the contents of the packaging to flow through the tubing, which is formed by the connection between the feeding set and the enteral feeding tube, reaching the specific region of the digestive system where it will be processed and absorbed.

Regarding the resolution of challenges that remain unsolved in the current prior art:

For enteral administration, products in aseptic cartons depend on one of two approaches present in the current prior art. The first approach relies on external devices, such as connectors and adapters, compatible with standard enteral feeding sets, as illustrated in patent documents CN1711116A, EP1352843A1, and WO2019193213A1. The second approach employs specialized feeding sets that replace the pointed ends of standard feeding sets with structures specifically designed for direct connection to the spouts of aseptic cartons, exemplified by prefabricated enteral administration systems, as shown in patent CN1711116A. In both cases, these are external devices that attach to the spouts provided by the opening devices of these packages, allowing administration via the enteral route. However, both approaches offer alternative auxiliary devices external to the packaging, with their methods of use revealing the challenges of administration in a closed enteral system.

It is widely recognized that enteral products manufactured in aseptic cartons are not provided with opening devices equipped with specific mechanisms for connecting standard enteral feeding sets.

It becomes clear that current practice does not provide an effective solution for administering enteral products in aseptic cartons that fully complies with the requirements of a closed enteral system.

The present invention overcomes the deficiencies observed in current technology by providing an innovative opening device in aseptic cartons. This device allows for the administration of enteral products manufactured in such cartons using standard enteral feeding sets. It stands out by eliminating the need for additional external devices and meeting the criteria established for closed enteral nutrition systems.

### DESCRIPTION OF THE DRAWINGS

The invention can be realized in various layouts with alternative mechanisms and complementary components. Therefore, a description of the figures is provided below to illustrate the invention and some of its concepts:

FIG. 1 presents a photograph of a traditional aseptic carton package (E) with an automated opening device (D), as commonly used in the current market. To the right, a representation of the same package (E) without the automated opening device (D) highlights the protective seal(S).

FIGS. 2 and 3 present the opening device (T) equipped with a device (4) for accessing an ENFit set, showing the cap (1) and the base (2) separated.

FIG. 4 illustrates the bottom view (a) and top view (b) of the base (2) of the opening device (T) equipped with a device (4) for ENFit set access and a biological filter (6).

FIG. 5 shows the bottom view (a) and the top view (b) of the base (2) with ENFit set access, without a biological filter.

FIG. 6 illustrates the opening device (T) equipped with a device (4) for ENFit set access. It differs in how the cap (1) is secured to the base (2) through a male thread (8) on the cylindrical body (2.4) of the base (2) and a corresponding female thread (9) on the cap (1).

FIG. 7 shows the opening device (T) equipped with a device (4) for ENFit set access. It differs in the type of closure, which is achieved by pressing a snap-on cap (10) onto the cylindrical body (2.2) of the base (2). The cap (10) is coupled to the base (2) via a foldable connection (11).

FIG. 8 shows the opening device (T) equipped with a device (12) for Spike set access, with the cap (1) and base (2) displayed separately.

FIG. 9 illustrates the bottom view (a) and top view (b) of the base (2) of the opening device (T), equipped with a device (12) for Spike set access, and additionally featuring a biological filter.

FIG. 10 shows the bottom view (a) and top view (b) of the base (2) of the opening device (T), equipped with a device (12) for Spike set access, without a biological filter.

FIG. 11 shows the opening device (T), equipped with a device (12) for Spike set access, differing in its closing method, which is achieved by pressing the attached cap (10) onto the cylindrical body (2.2) of the base (2). The cap (10) is attached to the base (2) via a foldable connection (11).

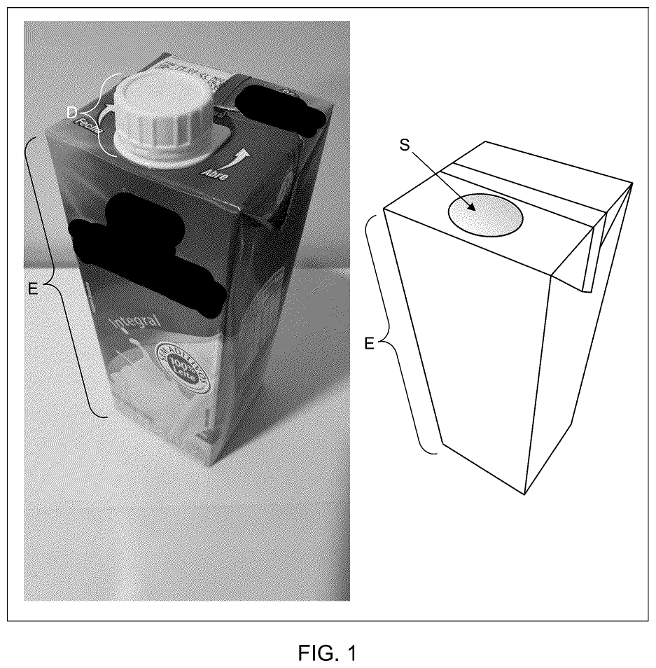

The most relevant components in the present invention and in an aseptic multilayer package (E) are described below:
(T)—An opening device for an aseptic multilayer package with access for an enteral feeding set in a closed system.
(E)—Aseptic multilayer carton packaging.
(S)—Protective seal of the aseptic multilayer packaging, comprising a circular pre-weakened area created by removing the upper layers of plastic and cardboard to create a zone without cutting resistance.
(C)—Packaging system consisting of an aseptic multilayer carton (E) and an opening device (T).
(1)—Cap, the upper part of the opening device (T).
(1.1)—Cylindrical structure positioned orthogonally to the interior face of the closing region (1.3) of the cylindrical body (1.2) of the cap (1), internally provided with a thread (7) corresponding to the thread (4.1) of the access device (4).
(1.2)—Cylindrical body that forms the cap (1).
(1.3)—Closing region at the upper edge of the cylindrical body (1.2) of the cap (1).
(2)—Base, the lower part of the opening device (T).
(2.1) Area on the bottom surface of the base (2) where hot-melt adhesive is applied, securing the opening device (T) permanently to the packaging (E).
(2.2)—Cylindrical body that forms the upper part of the base (2).
(2.3)—Flat body that forms the lower part of the base (2).
(2.4)—Elevated ring structure around the cylindrical body (2.2) of the base (2) that allows the cap (1) to fit by pressure.
(3)—Tamper-evident seal provided on the lower edge of the cap (1).
(3.1)—Structure on the base (2) for connection with the tamper-evident seal (3).
(4)—Access device located in the closing region (2.5) of the cylindrical body (2.2) of the base (2), specifically designed to connect ENFit sets.
(4.1)—Male thread on the access device (4), corresponding to the female thread found in the securing device of an ENFit set.
(4.2)—Circular region on the surface of the access device (4). This area is where the sealing ring of an ENFit set is compressed, ensuring a hermetic seal of the package.
(5)—Cross-shaped hole in the access device (4) for fitting and penetrating an ENFit set.
(6)—Biological filter located in the sealing region (2.5) of the cylindrical body (2.2) of the base (2), allowing air to enter the packaging.
(7)—Female thread inside a cylindrical structure (1.1) situated within the cylindrical body (1.2) of the cap (1), corresponding to the male thread (4.1) of the access device (4) in the base (2).
(8)—Male thread on the outer region of the cylindrical body (2.2) of the base (2), corresponding to a female thread (9) inside the cylindrical body (1.2) of the cap (1).
(9)—Female thread inside the cylindrical body (1.2) of the cap (1), matching the male thread (8) of the cap (1).
(10)—Cap attached to the base (2) via a hinge connection (11).
(11)—Hinge connection that joins the cap (10) to the base (2).
(12)—Cylindrical body forming the access device located in the sealing region (2.5) of the cylindrical body (2.2) of the base (2), specially designed to be penetrated by a Spike set. The sealing structure of the Spike set is compressed circumferentially within the inner region of this device (12), ensuring a hermetic seal of the packaging.
(12.1)—Sealing region at the lower edge of the cylindrical body (12) that forms the access device, made with a thinner thickness to create a weakened area to facilitate penetration by a Spike set.
(13)—Solid cylindrical body provided orthogonally to the inner face of the sealing region (1.3) of the cap (1). It consists of a device that fits snugly inside the access device (12) of the base (2).

### DESCRIPTION OF THE INVENTION

For visualization purposes, terms such as “superior”, “inferior”, “vertical”, and “horizontal” refer to positions in the Figures as depicted. “Longitudinal” and “transversal” refer to the viewing sequence, from top to bottom and from left to right, respectively. The term “anterior” should be related to what is visible in the Figure, and the term “posterior” should be related to what is on the opposite side of the Figure.

### About the Concept of the Invention:

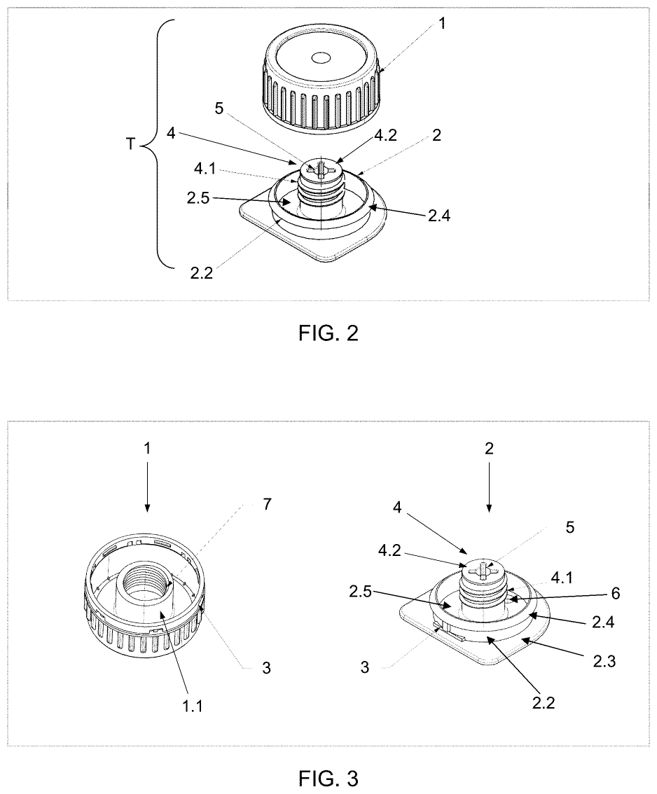

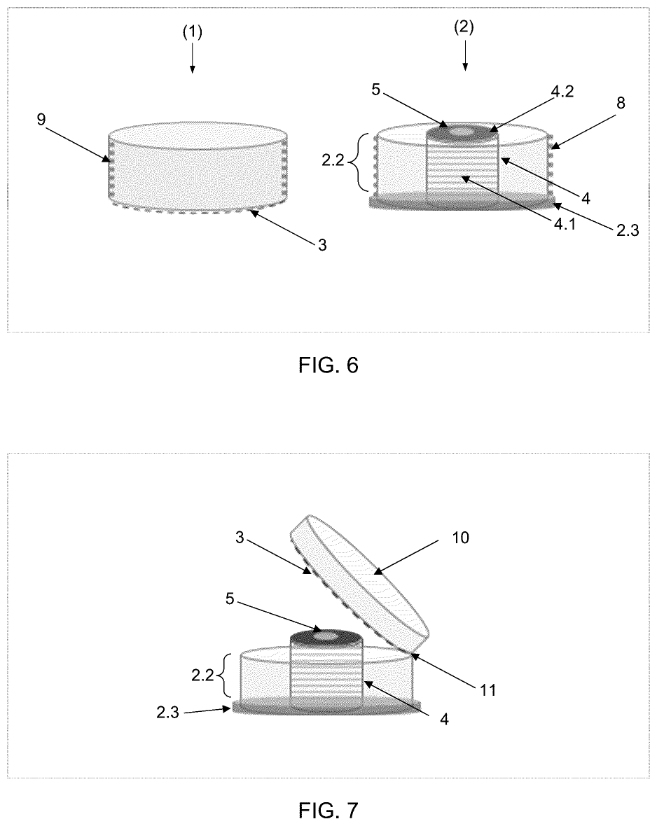

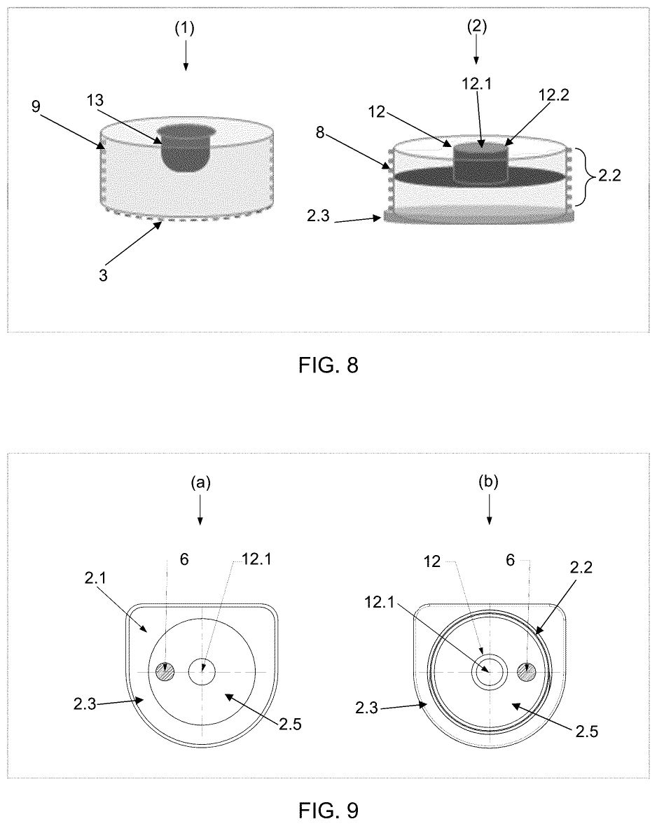

This opening device model (T) is designed so that aseptic multilayer cartons (E) can be used directly (eliminating the need for connectors/adapters) in closed enteral nutrition systems. As demonstrated in the Figures, it can be produced in various designs, either to fit different types of feeding sets available in the market or to facilitate production or improve handling experience. To illustrate compatibility with different models of enteral sets, the designs of the opening device (T) have been presented for connection with ENFit Sets (FIGS. 2, 3, 6, and 7) and with Spike Sets (FIGS. 8 and 11). The possibilities illustrated through various layouts can be applied with or without a biological air intake filter.

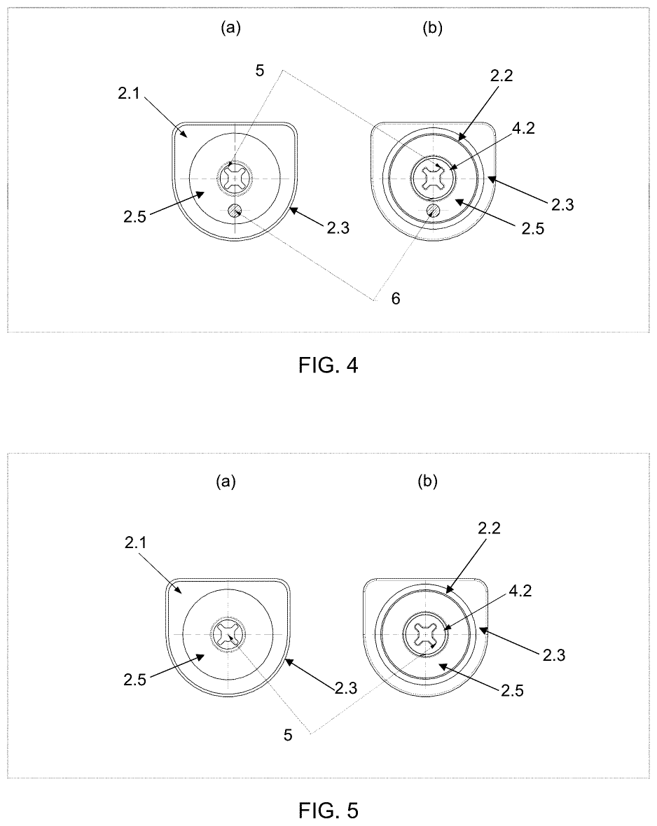

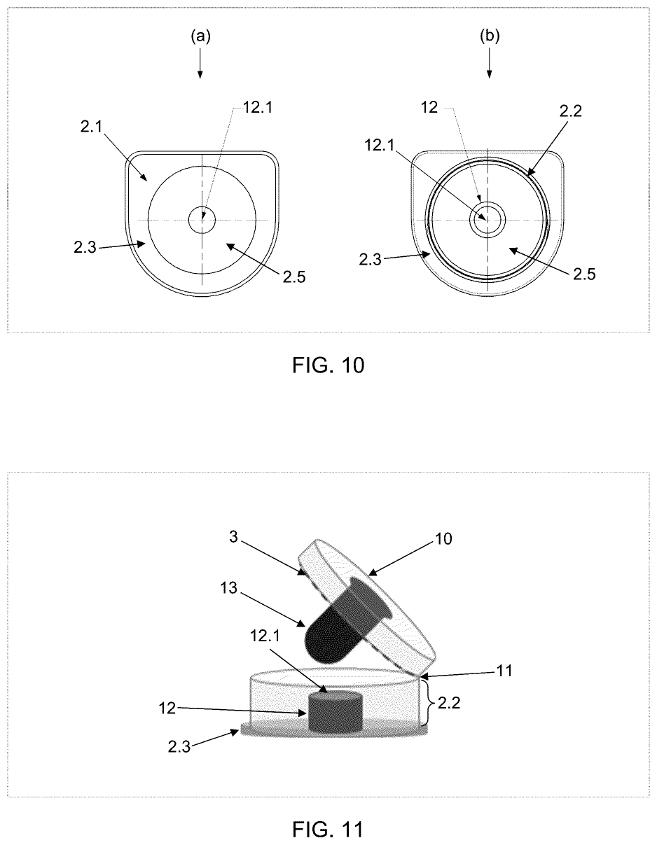

Despite having a structure based on the opening devices predominantly found in aseptic cartons, this opening device (T) possesses technical features typically found in special packaging for enteral products intended for administration through a closed system: access devices (4 and 12) for enteral sets, whether ENFit type, Spike type, or other existing types; and a microbiological filter (6), which is unnecessary in packages designed to collapse during product administration. Illustrative diagrams have been presented with a filter (FIGS. 4 and 9) and without a filter (FIGS. 5 and 10), for both models with an access device (4) for ENFit sets and models with an access device (12) for Spike sets.

The inventive concept of the opening device (T) is illustrated in FIG. 2 as “an opening device (T) for aseptic carton packaging (E) equipped with an access device (4) for an enteral set at its base (2).” This device (T) is composed of two essential parts: a cap (1) and a base (2).

The cap (1) consists of a cylindrical body (1.2), closed at the top (1.3), and can feature a tamper-evident seal (3) on its lower edge to show that the product has not been opened. The cap (1) has a fitting mechanism, preferably an internal thread (9) in its cylindrical structure. Another fitting mechanism can be achieved, for instance, through a thread (7) inside a cylindrical structure (1.1) provided within the cap (1) to couple with the access device (4) in the base (2). The fitting can also occur via a snap-fit system (2.4) or by a combination of both (2.4 and 7), as illustrated in FIG. 3. This configuration ensures effective closure of the cap (1) over the base (2). Instead of the cap (1), the opening device (T) can provide an attached cap (10) connected to the base (2) via a foldable connection (11).

The base (2) can be better understood when divided into two elements: the lower element (2.3) and the upper element (2.2). The lower element (2.3) of the base (2) includes a substantially flat body in its upper region, while its lower region (2.1) is designed to receive a portion of hot-melt adhesive that irreversibly attaches the device (T) to the aseptic carton (E) over the seal(S). The upper element of the base (2) includes a cylindrical body (2.2) that is substantially perpendicular to the lower element (2.3) and may contain fitting mechanisms (2.4, 8) corresponding to the fitting mechanisms (9) in the cap (1). Unlike existing opening devices in aseptic cartons, this cylindrical body (2.2) contains a closing region (2.5) internally, which is equipped with an access device (4; 12) for connecting an enteral set and may also include a biological filter (6).

### About the Construction of the Invention:

The opening device (T) is structured in two main parts: the cap (1) and the base (2). The manufacture of these components is predominantly achieved through the plastic injection molding process. In this process, selected thermoplastic resins such as polypropylene or polyethylene are heated until reaching a fluid state. They are then injected under controlled pressure into high-precision molds.

To achieve the desired robustness and specificity, these parts can be made from a combination of sub-parts, also resulting from the plastic injection molding process. The joining of these sub-parts can utilize a range of techniques, from precise and irreversible press-fit connections to methods such as hot welding (thermofusion), ultrasonic welding, vibration welding, laser welding, or any other method available in the plastics industry.

As with the caps of non-collapsible packages for closed-system enteral products, some types of this opening device (T) are equipped with a microbiological filter (6). This filter (6) is essential in non-collapsible packages because it ensures that properly filtered air enters the package's interior, providing the right pressure balance and guaranteeing a continuous flow during the infusion of the enteral product into the patient. The integration of this filter (6) into the closing region (2.5) of the base (2) of the opening device (T) can be achieved through plastic welding, adhering the filter (6) directly to the base (2) in a stable manner. Alternatively, the filter (6) can be placed between plastic parts that, when press-fitted, become irreversibly joined, or through other plastic welding methods mentioned earlier.

Once the molding and assembly processes of the key parts are complete, they are joined to form a single device (T). This process, which may involve techniques like threading and/or press-fits, ensures the integrity of the opening device (T). The result is a robust, harmonized, and highly functional structure, ready to be integrated into the aseptic carton, guaranteeing a securely sealed and safe product.

### EXAMPLES OF EMBODIMENTS OF THE INVENTION

Some examples of embodiments of the invention are represented in FIGS. 2, 3, 6, 7, 8, and 11. It is worth highlighting that, due to its ease of application in aseptic cartons, such a device (T) can be implemented in any production line equipped with an aseptic filling machine and standard device (D) applicator machines.

1. An opening device integrated with a multilayer aseptic carton package used for closed-system enteral nutrition, where the device comprises:
a cap consisting of a cylindrical body closed at the top and equipped with an external seal on its lower edge, with an internal thread provided inside the cylindrical body;
a base consisting of a lower element and an upper element, wherein:
the lower element of the base comprises a substantially flat body in its upper region, while its lower surface can receive an amount of adhesive required to attach the device to the aseptic carton package;
the upper element of the base comprises a cylindrical body that is substantially perpendicular to the lower element; the cylindrical body of the base is equipped with a mechanism to enable coupling of the cap to the base;
the device is characterized by:
an internal section of the cylindrical body of the base being provided with a circular closing region; and
an access device for connecting an enteral feeding set, which is provided in the closing region of the base.

2. The opening device according to claim 1, characterized by a biological filter being additionally provided in the aforementioned closing region of the base.

3. The opening device according to claim 1, characterized by providing a foldable connection that joins the coupled cap to the base.
